(19) United States
(12) Patent Application Publication
(10) Pub. No.: US 2004/0219118 A1
(43) Pub. Date: Nov. 4, 2004

(54) Method and kit for reducing irritation of skin depilatory compositions

(75) Inventors: Craig Stephen Slavtcheff, Guilford, CT (US); Jessica Weiss Goldberg, Fairfield, CT (US); Anat Shiloach, Norwalk, CT (US); Michael Massaro, Monroe, CT (US); Christine Elizabeth Kennedy, Torrington, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/669,547

(22) Filed: Sep. 24, 2003

Related U.S. Application Data
(60) Provisional application No. 60/467,628, filed on May 2, 2003.

(51) Int. Cl. A61K 7/06

(52) U.S. Cl. 424/70.1

(57) ABSTRACT

A method and kit is provided for removing hair. The method includes applying to an area of skin from which hair is to be removed a skin pretreatment composition that includes a lipophilic material. Thereafter applied onto the pretreated area is a depilatory composition including a keratin degrading agent in an effective amount to chemically react with hair to allow wipe-away removal. A kit is also provided which includes a skin pretreatment product formed with a skin pretreatment composition including a lipophilic material delivered to the skin by a carrier. The kit further includes a depilatory product having a depilatory composition with a keratin degrading agent chemically reactive with hair to weaken the hair for removal, the depilatory composition being delivered from a carrier.

### BACKGROUND OF THE INVENTION

1. Field of the Invention

The invention concerns a method and kit for reducing irritation and other forms of skin damage often associated with depilation, particularly on sensitive skin.

2. The Related Art

Depilatories are cosmetic composition which remove hair through chemical reaction by active agents. Predominantly these agents sever disulfide bonds. This weakens hair to such an extent that subsequent scraping and wiping away of the depilatory composition completes severance of hair at the skin surface and allows removal.

Commercially the most common agents are thioglycolates typically formulated at high pH. Effectiveness requires prolonged residence time on the skin, generally for greater than four minutes. These conditions for chemical reaction are relatively aggressive. Significant irritation and inflammatory response can arise from the procedure, particularly experienced by those with sensitive skin.

The art has tried to mitigate irritation attributable to chemical irritants. For instance, U.S. Pat. No. 6,139,850 (Hahn et al.) utilizes aqueous-soluble strontium salts as anti-irritants. U.S. Pat. No. 5,756,107 (Hahn et al.) identifies other alkali metal cations such as aqueous-soluble lithium or potassium salts as effective for the same purpose.

Some patents describe depilatory formulations that reduce irritation by minimizing contact time. For example, GB 1 329 029 (Colgate-Palmolive) describes a self-heating depilatory in which the thioglycolic acid is packaged separately from the base. Heat that is generated upon their combination speeds the depilation reaction. Contact times are thereby shortened.

WO 02/43682 A1 (Kovacs et al.) discloses a product for shaving and depilation with benefits in softening hair, keeping the skin calmed and having a discrete scent. These benefits are achieved through a homogenized product consisting of greasy oils, essential oils, antioxidant additives and preferably an essence of perfume.

Another approach is disclosed in U.S. Pat. No. 6,306,380 B1 (Desmots et al.). Cosmetic depilatory compositions are described with a continuous aqueous phase and an oil phase. Partition of the keratin-degrading substance is formulated to distribute itself both in the aqueous and oil phases. When the keratin-degrading substance in the continuous aqueous phase has been consumed, the remainder in the oil phase becomes available in a regulated release thereby resulting in a lower irritancy.

None of the aforementioned approaches have been completely successful. Further improvements are needed to achieve a depilatory both functionally effective and sufficiently mild for even sensitive skin.

### SUMMARY OF THE INVENTION

A method for removing hair is provided which includes:

(i) applying to an area of skin from which hair is to be removed a skin pretreatment composition that includes a lipophilic material; and

(ii) applying onto the pretreated area of skin a depilatory composition including a keratin degrading agent in an effective amount to chemically react with hair to allow wipe-away removal.

Furthermore, there is provided a kit for removal of hair from an area of skin which includes:

(a) a skin pretreatment product which includes:

(i) a skin pretreatment composition that includes a lipophilic material; and

(ii) a carrier for delivering the skin pretreatment composition onto the area of skin;

(b) a depilatory product which includes:

(i) a depilatory composition including a keratin degrading agent in an effective amount to chemically react with hair to allow removal; and

(ii) a carrier for delivering the depilatory composition onto the area of skin subsequent to delivery of the pretreatment composition;

(c) instructions to apply the skin pretreatment product to an area of skin requiring removal of hair followed by an application of the depilatory product over the area of skin covered by the pretreatment product.

Instructions normally will be in written form on packaging for the kit, on one or both of the pretreatment and depilatory products, or on a separate written instruction insert within the kit package.

### DETAILED DESCRIPTION OF THE INVENTION

Now it has been found that the irritation associated with chemical depilation of hair can be reduced or eliminated without loss of efficiency.

Pretreatment of the depilation area with lipophilic materials significantly protects skin from redness/erythema. Despite an underlying layer of the lipophilic material, chemical reaction by the keratin degrading actives remains unimpeded. The desired hair removal is achieved without extending residence time of the actives.

Lipophilic materials of the present invention are not limited to but may preferably include oils such as hydrocarbons, natural or synthetic esters and silicones. Among the hydrocarbons suitable examples are mineral oil, isoparaffins, petroleum jelly (petrolatum), polymerized olefins (e.g. polybutenes, polydecenes, isohexadecane) and combinations thereof.

Illustrative of the natural esters are vegetable oils such as sunflower seed oil, cottonseed oil, rapeseed oil, safflower oil, olive oil, borage oil, borage seed oil, soybean oil and combinations thereof.

Among the synthetic esters are alkenyl esters of fatty acids having from 10 to 20 carbon atoms (e.g. methyl myristate, methyl stearate, oleyl stearate, butyl oleate, isopropyl myristate, isopropyl palmitate and combinations thereof); sterol esters (such as cholesterol fatty acid esters), ether-esters (such as fatty esters of ethoxylated fatty alcohols), wax esters (such as beeswax, spermaceti and candilla) and mixtures thereof.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms.

Linear volatile silicone materials generally have viscosities less than about 5 centistokes at 25° C. while cyclic materials typically have viscosities of less than about 10 centistokes.

Nonvolatile silicone oils useful as an emollient material include polyalkyl siloxanes, polyalkylaryl siloxanes and polyether siloxane copolymers (e.g. dimethicone copolyol). The essentially non-volatile polyalkyl siloxanes useful herein include, for example, polydimethyl siloxanes with viscosities of from about 5 to about 100,000 centistokes at 25° C. Among the preferred non-volatile emollients useful in the present compositions are the polydimethyl siloxanes having viscosities from about 10 to about 400 centistokes at 25° C.

The lipophilic material may range in amount from 1 to 100% by weight of the skin pretreatment composition, preferably in amounts from about 30 to about 98%, more preferably from about 60 to about 90% by weight of the skin pretreatment composition.

Keratin degrading agents normally are those materials capable of reducing the disulfide linkages found in keratin. Illustrative but non-limiting examples of such agents are potassium thioglycolate, dithioerythritol, thioglycerol, thioglycol, thioxanthine, thiosalicyclic acid, N-acetyl-L-cysteine, lipoic acid, sodium bisulphide, dilithium sulphide, disodium sulphide, dipotassium sulphide, magnesium sulphide, calcium sulphide, strontium sulphide, barium sulphide, diammonium sulphide, sodium dihydrolipoate 6,8-dithioctanoate, sodium 6,8-dithiooctanoate, salts of hydrogen sulphide such as sodium mercaptan or potassium mercaptan, thioglycolic acid, 2-mercaptopropionic acid, 3-mercaptopropionic acid, thiomalic acid, ammonium thioglycolate, glyceryl monothioglycolate, monoethanolamine thioglycolate, diammonium dithiodiglycolate, ammonium thiolactate, monoethanolamine thiolactate, thioglycolamide, homocysteine, cysteine, glutathione, dithiothreitol, dihydrolipoic acid, 1,3-dithiopropanol, glycerylmonothioglycolate, thioglycolhydrazide, keratinase, hydrazine sulphate, hydrazine disulphate, triisocyanate, guanidine thioglycolate, calcium thioglycolate and/or cysteamine. Most preferred are the thioglycolates, especially the salts of sodium, potassium and calcium thioglycolates and mixtures of these salts. Amounts of the keratin degrading agent may range from about 0.5 to about 15%, preferably from about 1 to about 10%, optimally from about 2 to about 8% by weight of the depilatory composition.

Optionally, the depilatory composition may include an accelerator for speeding the keratin degradation reaction. Illustrative accelerators include urea, thiourea, dimethyl isosorbide (DMI), ethoxydiglycol (Transcutol) or methyl propyl diol (MP diol). Preferred are urea or methyl propyl diol. These accelerators may be present in amounts from about 0.5 to about 25%, preferably from about 1 to about 12%, optimally from about 5 to about 10% by weight of the depilatory composition.

It is particularly preferred that the depilatory composition include a pH regulator. The quantity and type of pH regulator should be chosen to maintain the pH at a value greater than 5, preferably greater than 7, more preferably in the range from about 8 to about 13, optimally in the range from about 11.5 to about 12.5, particularly about 12.

Examples of pH regulators include alkaline and alkaline earth hydroxides (sodium, potassium, lithium and calcium hydroxides), alkaline silicates (such as meta-or trisilicates), L-arginine, polyethyleneimine and combinations thereof. Amounts of the pH regulator may range from about 0.1 to about 5%, preferably from about 0.5 to about 3% by weight of the depilatory composition.

Thickeners/viscosifiers may also be present in the depilatory composition in amounts from about 0.1 to about 100%, preferably from about 0.3 to about 3% by weight. Exemplary thickeners are xanthan gum, sodium carboxymethyl cellulose, hydroxyalkyl and alkyl celluloses (particularly hydroxypropyl cellulose), polyacrylamide (such as found in Sepigel 305®), Sclerotium gums, magnesium aluminum silicates sold as Veegum®, Carbomers (such as Carbopol® 980) and aluminum octenyl starches such as Dry Flo®.

Surfactants may also be employed in the depilatory compositions of the present invention. They may be of the cationic, anionic, nonionic or amphoteric variety. Nonionic surfactants include alkoxylated fatty alcohols, alkoxylated fatty acids and alkoxylated sorbitan esters. They also may include alkyl polyglycosides and gluconamides. Anionic surfactants may include alkyl sulphates, alkyl ether sulphates, acyl isethionates, lactylates, sarcosinates, taurates and combinations thereof. Suitable amphoteric surfactants include cocoamidopropyl betaine and dimethyl alkyl amine oxides. Preferred surfactants generally are nonionic and may include cetearyl phosphate, cetearyl alcohol, myristyl alcohol, cetearyl glucoside, cetearyl alcohol ethoxylates and combinations thereof. Amounts of the surfactant may range anywhere from about 0.1 to about 10%, preferably from about 1 to about 5% by weight of the depilatory composition.

Humectants of the polyhydric alcohol-type may also be included in the depilatory compositions of this invention. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, isoprene glycol, hexylene glycol, 1,3-butylene glycol, 1,2,6-hexanetriol, ethoxylated glycerol, propoxylated glycerol and mixtures thereof. The amount of humectant may range from about 0.5 to about 30%, preferably between 1 and 15% by weight of the depilatory composition.

Colorants and fragrances may be included in depilatory or skin pretreatment compositions of the present invention. Each of these substances may range from about 0.05 to about 5%, preferably between about 0.1 and about 30% by weight for the respective compositions.

Depilatory compositions are generally emulsions. These may be of the oil-in-water or water-in-oil type. Amounts of water may range from about 20 to about 98%, preferably from about 50 to about 90%, optimally from about 75 to about 85% by weight of the depilatory compositions. Oils present in the emulsion may be mineral oils, isoparaffins, petroleum jelly, silicones or combinations thereof. Amounts of oil may range from about 2 to about 60%, preferably from about 10 to about 40%, optimally from about 15 to about 25% by weight of the depilatory composition.

The depilatory and skin pretreatment compositions will be delivered by a carrier. For purposes of this invention, the term “carrier” is considered to be a mechanical water-insoluble rigid or flexible plastic or fibered device. Illustrative devices for use as carriers include jars, aerosol or mechanical spray pump devices, roll-on ball dispensers, propel-repel stick dispensers (similar to those used in antiperspirant sticks) and flexible woven or non-woven water-insoluble textiles. When the carrier is a woven or non-woven flexible textile, this may be in the form of a glove or a single or multi-layered cloth. The textile may be textured or non-textured. It may be apertured or non-apertured. One surface of the wipe may contain the depilatory composition and the other the skin pretreatment composition.

The carrier may be a single or multi-compartment device. When the carrier is a single device, preferably it is a tube where each of the depilatory and skin compartment pretreatment compositions remain separate from one another. Illustrative is a depilatory and skin pretreatment composition each in the form of a semi-viscous material.

Therein the compositions are arranged adjacent one another and extruded from the single tube package as two separate stripes. Illustrative of this technology is U.S. Pat. No. 4,211,341 (Weyn) herein incorporated by reference.

Carriers for the respective depilatory and skin pretreatment compositions may be in the form of dual-compartment dispensers. Illustrative is a dual pump as shown in U.S. Pat. No. 5,740,947 (Flaig et al.) herein incorporated by reference. The carriers for each of the compartments may also be in the form of stackable jars such as disclosed in U.S. Pat. No. 5,914,116 (Suares et al.) herein incorporated by reference.

Carrier packages for the respective compositions may be different from one another. For instance, there may be combinations of a roll-on dispenser with a jar, a textile with a roll-on dispenser, a textile with a jar, a spray nozzle pump with a jar, a textile with a spray nozzle pump, a glove with a jar, a glove with a spray nozzle pump dispenser, or combinations thereof.

Kits according to the present invention may constitute a combination of the aforementioned modes of packaging, with each of the respective packages containing one of the respective compositions.

The depilatory and skin pretreatment compositions may be applied from their respective carriers onto the skin requiring depilation in equal or unequal amounts. The weight ratio of the applied respective compositions may range from about 100:1 to about 1:100, preferably from about 20:1 to about 1:20, and optimally from about 2:1 to about 1:2 by weight.

Ordinarily the kits will be supplied with a set of instructions to first apply the pretreatment composition and thereafter the depilation composition. Instructions on time between the applications may also be provided. Dependent upon the particular compositions, there may be no waiting time or as much as thirty minutes between applications of the two different compositions.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following examples will more fully illustrate the embodiments of this invention. All parts, percentages and proportions referred to herein and in the appended claims are by weight unless otherwise illustrated.

### EXAMPLE 1

Eight panelists evaluated a commercially available depilatory composition alone and in combination with a pretreatment composition of mineral oil. The pretreatment and non-pretreatment applications were applied to one each of the respective legs of every panelist.

Each panelist on one of their legs spread mineral oil over the target depilation area and left this on for five minutes. Thereafter, the depilatory composition was spread over the pretreatment area and also on a non-pretreated area of the other leg. After 10 minutes, both areas were rinsed with warm water to rinse-off the depilatory composition and mineral oil where present and to remove severed hair.

The depilation composition utilized for the test is a formulation reported in Table I below.

TABLE I

| INGREDIENT | WEIGHT % |
|---|---|
| Water | 84.5 |
| Thioglycolic Acid | 3.0 |
| Hydroxide Salt (Ca, Na, K) | 4.2 |
| Mineral Oil | 4.0 |
| Cetearyl Alcohol and Ceteareth-20 | 4.3 |

Results of the test are reported in Table II below. They reveal that efficacy was essentially the same with the pretreatment and non-pretreatment regimes. However, the pretreatment regime was substantially preferred for having less irritation, leaving legs feeling smooth, leaving legs moisturized and was better in the application process.

TABLE II

| | Preference | | |
|---|---|---|---|
| | No Pretreat | Pretreat | No Pref. |
| Removed Hair | 1 | 2 | 5 |
| Had Less Irritation | 2 | 4 | 2 |
| Leaves Legs Feeling Smooth | 1 | 3 | 4 |
| Leaves Legs Moisturized | 1 | 4 | 3 |
| Had Better Application Process | 1 | 2 | 5 |
| Prefer Overall | 1 | 5 | 2 |

### EXAMPLE 2

Theoretically separation of the depilation composition by the pretreatment layer from the target skin should inhibit effectiveness of the keratin degrading agent. However, the consumer panel test reported in Example 1 did not reveal any difference in hair removal effectiveness with or without the pretreatment.

A test in the form of a clinical was conducted to further evaluate efficacy. Objective for the clinical was to explore what effect a mineral oil pretreatment would have on the efficacy of a standard depilatory composition. Sixteen panelists were chosen for the study.

Each panelist had two 5×5-cm sites marked on each outer lower leg. Baseline evaluations (Charmview Videomicroscope) were made prior to product application. Two of the four test sites were treated 5 minutes prior to product application with 0.04 ml of mineral oil. Then 1.0 g of depilatory product was applied to each test site, and was exposed for 10 minutes. Each test site was then wiped clean using a damp paper towel. Readings were then taken thirty minutes after removal.

The commercial product whose formula is identified in Table I was again utilized for this clinical. Mineral oil was employed as the pretreatment composition.

Visual grading was conducted using a five-point scale for erythema; the scale ranges from 0 (none) to 4 (severe). In addition, the Charmview Video microscope was utilized to measure redness on the forearms, and on the legs, and the images were analyzed to determine percent removal of hair. The analysis was done with Optimas Image Analysis software.

Within each ROI (“Region of Interest”) the hair fibers were threshold of density measured and at that threshold measured for pixel length. The mean sum of hair fibers was then determined and compared post treatment to determine percent removal of hair.

The percent decrease in mean hair length for the non-pretreated application was 88%. The pretreatment application had a percent decrease in mean hair length of 77%. The difference between these mean hair length results was relatively small considering that the hair targets were first shielded with mineral oil and were not expected to allow adequate penetration of the depilation composition.

### EXAMPLE 3

This Example illustrates use of a glove as a carrier for the skin pretreatment composition in the context of the present invention. A latex rubber glove is coated with mineral oil and sealed in a plastic pouch.

When ready for use, the pouch is opened and the user places their hand in the glove with the oil coated surface on the outside. The oil is then spread on the area of the leg and other body parts intended for the depilation. After a five minute delay, a depilation composition held within a roll-on ball dispenser is applied over the mineral oil pretreated skin. After ten minutes, the pretreatment and depilation compositions are wiped away with a paper towel. Successful hair removal is achieved. The depilation composition is described in Table III below.

TABLE III

| INGREDIENT | WEIGHT % |
|---|---|
| Cetostearyl Alcohol | 8.0 |
| Sodium Magnesium Silicate | 1.0 |
| Calcium Hydroxide | 0.5 |
| Urea | 8.0 |
| L-Arginine | 2.0 |
| Polyethylenimine | 1.0 |
| Magnesium Trisilicate | 0.5 |
| Titanium Dioxide | 0.4 |
| Potassium Thioglycolate (30% Active) | 10.0 |
| Shea Butter | 0.5 |
| Fragrance | 0.5 |
| Paraffin Oil | 3.5 |
| Propylene Glycol | 0.3 |
| Acrysol 33 ® (Acrylic Copolymer) | 0.01 |
| Arlamol E ® | 1.0 |
| Ceteareth-20 | 3.0 |
| Deionized Water | Qs |

### EXAMPLE 4

This Example illustrates a kit based upon a carrier which is a dual nozzle non-simultaneous delivery pump as shown in U.S. Pat. No. 5,740,947 (Flaig et al.). In one of the chambers, a 100% sunflower seed oil pretreatment composition is stored. In the other chamber a depilation composition is stored having the formula shown in Table IV below.

TABLE IV

| INGREDIENT | WEIGHT % |
|---|---|
| Cetearyl Alcohol | 10.0 |
| Calcium Thioglycolate | 7.0 |
| Calcium Hydroxide | 5.0 |
| Mineral Oil | 3.0 |
| Tetrasodium EDTA | 0.5 |
| Sodium Laurylethersulphate (27% Active) | 0.5 |
| Water | qs |

With the present kit there is included a polypropylene non-woven high loft textile. The vegetable oil is expressed from the pump onto the textile. A body part requiring depilation is then coated with the sunflower seed oil by wiping with the textile. After a one minute wait, the depilation composition of Table IV is expressed from the other compartment of the pump onto a second non-woven textile. This composition is then applied over the sunflower seed oil pretreated area. After a period of twenty minutes, both compositions are removed by wiping with a third non-woven textile to remove the unwanted hair.

What is claimed is:

1. A method for removing hair comprising:
(i) applying to an area of skin from which hair is to be removed a skin pretreatment composition that comprises a lipophilic material; and
(ii) applying onto the pretreated area of skin a depilatory composition comprising a keratin degrading agent in an effective amount to chemically react with hair to allow removal.

2. The method according to claim 1 wherein the lipophilic material is selected from the group consisting of hydrocarbons, natural or synthetic esters, silicones and mixtures thereof.

3. The method according to claim 1 wherein the lipophilic material is mineral oil.

4. The method according to claim 1 wherein the keratin degrading agent is a thioglycolate salt.

5. The method according to claim 1 wherein the skin pretreatment composition is applied for at least about one minute prior to application of the depilatory composition.

6. The method according to claim 1 wherein the skin pretreatment composition is applied for at least five minutes prior to applying the depilatory composition.

7. A kit for removal of hair from an area of skin comprising:
(a) a skin pretreatment product comprising:
(i) a skin pretreatment composition that comprises a lipophilic material; and
(ii) a carrier for delivering the skin pretreatment composition onto the area of skin;
(b) a depilatory product comprising:
(i) a depilatory composition comprising a keratin degrading agent in an effective amount to chemically react with hair to allow wipe away removal; and
(ii) a carrier for delivering the depilatory composition onto the area of skin subsequent to delivery of the pretreatment composition;
(c) instructions to apply the skin pretreatment product to an area of skin requiring removal of hair followed by an application of the depilatory product over the area of skin covered by the pretreatment product.

8. The kit according to claim 7 wherein the carrier for delivering the skin pretreatment composition is a textile.

9. The kit according to claim 8 wherein the textile is a glove.

10. The kit according to claim 7 wherein the depilatory and skin pretreatment compositions are applied from their respective carriers in weight ratios ranging from about 100:1 to about 1:100.
